SEQUENCE LISTING (31 sequences)

SEQ ID NO: 1 - DNA, 20 bp, Artificial Sequence
  Synthetic Construct
cgctgatgaaaccggaaatg

SEQ ID NO: 2 - DNA, 21 bp, Artificial Sequence
  Synthetic Construct
gcttgtggcactcttgttatg

SEQ ID NO: 3 - DNA, 20 bp, Artificial Sequence
  Synthetic Construct
cgttggcgcagtttgtaaag

SEQ ID NO: 4 - DNA, 20 bp, Artificial Sequence
  Synthetic Construct
ccactcgcatttgagaggaa

SEQ ID NO: 5 - DNA, 22 bp, Artificial Sequence
  Synthetic Construct
gatctagcttggtgggagaaag

SEQ ID NO: 6 - DNA, 20 bp, Artificial Sequence
  Synthetic Construct
tgtatgtcgggtggtctttg

SEQ ID NO: 7 - DNA, 22 bp, Artificial Sequence
  Synthetic Construct
catagttgctgagttcgtaggg

SEQ ID NO: 8 - DNA, 22 bp, Artificial Sequence
  Synthetic Construct
gctttgaaagtgatggcgtaaa

SEQ ID NO: 9 - DNA, 19 bp, Artificial Sequence
  Synthetic Construct
taatacgactcactatagg

SEQ ID NO: 10 - DNA, 19 bp, Artificial Sequence
  Synthetic Construct
atgggtaaaggagaagaac

SEQ ID NO: 11 - DNA, 20 bp, Artificial Sequence
  Synthetic Construct
atcctgttgacgagggtgtc

SEQ ID NO: 12 - DNA, 24 bp, Artificial Sequence
  Synthetic Construct
tacgaacattaacgggaacgacgg

SEQ ID NO: 13 - DNA, 25 bp, Artificial Sequence
  Synthetic Construct
ggtatagctgggttcaccttcagtg

SEQ ID NO: 14 - DNA, 21 bp, Artificial Sequence
  Synthetic Construct
cagtggcaaatcatcaatgcg

SEQ ID NO: 15 - DNA, 24 bp, Artificial Sequence
  Synthetic Construct
atgtggagatttatttacagccag

SEQ ID NO: 16 - DNA, 23 bp, Artificial Sequence
  Synthetic Construct
gatctagcttggtgggagaaagg

SEQ ID NO: 17 - DNA, 20 bp, Artificial Sequence
  Synthetic Construct
ggggagcgaaaaatcggaga

SEQ ID NO: 18 - DNA, 22 bp, Artificial Sequence
  Synthetic Construct
ttcgtagggactttgctgttag

SEQ ID NO: 19 - DNA, 23 bp, Artificial Sequence
  Synthetic Construct
cggattgatgtgacaaccactaa

SEQ ID NO: 20 - DNA, 22 bp, Artificial Sequence
  Synthetic Construct
gaaactcgctcctcttgtagtt

SEQ ID NO: 21 - DNA, 23 bp, Artificial Sequence
  Synthetic Construct
tgtctgctcttcctgtcttattc

SEQ ID NO: 22 - DNA, 21 bp, Artificial Sequence
  Synthetic Construct
cgagtgcacgagtagtgtaaa

SEQ ID NO: 23 - DNA, 21 bp, Artificial Sequence
  Synthetic Construct
cacacccacatcagaggtaaa

SEQ ID NO: 24 - DNA, 20 bp, Artificial Sequence
  Synthetic Construct
gactggtatgctgctctccc

SEQ ID NO: 25 - DNA, 20 bp, Artificial Sequence
  Synthetic Construct
catctgaagcatgtgcagcc

SEQ ID NO: 26 - DNA, 20 bp, Artificial Sequence
  Synthetic Construct
ggagccatctgtggagcaat

SEQ ID NO: 27 - DNA, 20 bp, Artificial Sequence
  Synthetic Construct
agtgcttctatcgccacacc

SEQ ID NO: 28 - DNA, 22 bp, Artificial Sequence
  Synthetic Construct
taaattcgactcgactcacggt

SEQ ID NO: 29 - DNA, 20 bp, Artificial Sequence
  Synthetic Construct
ctccaccacatactcggctc

SEQ ID NO: 30 - DNA, 19 bp, Artificial Sequence
  Synthetic Construct
atgggtaaaggagaagaac

SEQ ID NO: 31 - DNA, 20 bp, Artificial Sequence
  Synthetic Construct
atcctgttgacgagggtgtc

(19) United States
(12) Patent Application Publication
(10) Pub. No.: US 2023/0210106 A1
(43) Pub. Date: Jul. 6, 2023

(54) RNAi FOR CONTROL OF PHLOEM SAP-FEEDING INSECTS IN CROP PLANTS

(71) Applicants: Cornell University, Ithaca, NY (US); Boyce Thompson Institute for Plant Research Inc., Ithaca, NY (US)

(72) Inventors: Angela SEARLE, Ithaca, NY (US); Edward Guy Robert TURGEON, Ithaca, NY (US); Joyce VAN ECK, Ithaca, NY (US)

(21) Appl. No.: 17/744,927

(22) Filed: May 16, 2022

Related U.S. Application Data
(63) Continuation of application No. 16/119,537, filed on Aug. 31, 2018, now abandoned.
(60) Provisional application No. 62/552,781, filed on Aug. 31, 2017.

(51) Int. Cl. A01N 25/00; C12N 15/82; C12N 15/113; A01N 65/40; A01N 57/16

(52) U.S. Cl. CPC A01N 25/006; A01N 57/16; A01N 65/40; C12N 15/113; C12N 15/8218; C12N 15/8286; C12N 2310/14

(57) ABSTRACT

A method involving administering to a phloem sap-feeding insect one or more dsRNAs capable of suppressing activity of one or more RNAi-suppressing nuclease genes expressed in the gut of the insect and one or more dsRNAs capable of suppressing one or more osmoregulatory genes expressed by the insect.

### CROSS-REFERENCE TO RELATED APPLICATIONS

This application claims benefit of U.S. Provisional Application No. 62/552,781, filed Aug. 31, 2017, which is hereby incorporated by reference in its entirety.

### FIELD OF THE INVENTION

The invention relates generally to methods of molecular biology and gene silencing to control insect pests in agriculture.

### BACKGROUND OF THE INVENTION

RNA interference (RNAi) has been gaining attention as a loss-of-function research tool. It shows great potential as a novel biological control mechanism for the protection of crops against different pests. Its intracellular mode of action is highly conserved and well described. The inherent sequence-specific nature of the mechanism allows for selectively targeting organisms, such as insect pests, by optimizing dsRNA fragments corresponding to species-specific gene sequences. RNAi technology, allowing for in vivo post-transcriptional silencing of essential genes, thereby causing mortality with little effect on nontarget species, has gained significant interest in pest management research over the past years.

RNA interference (RNAi) holds great promise as a novel strategy against insect pests of agricultural crops. This is because, in principle, the only information needed to target an essential insect gene with exquisite specificity is the insect gene sequence; and the RNAi molecules can be delivered via transgenesis of the crop plant. Specifically, the plant is engineered to express double-stranded RNA (dsRNA) against the insect gene of interest. On ingestion by the insect, the dsRNA is internalized into cells, where it is cleaved by an insect dsRNA-specific enzyme, Dicer-2, into small interfering RNA (siRNA, ca. 21 nt) that guides the Argonaute protein of the RNA induced silencing complex (RISC) to degrade complementary mRNAs. In planta RNAi has yielded significant plant protection against the western corn rootworm Diabroticavirgiferavirgifera, cotton bollworm Helicoverpaarmigera, and Colorado potato beetle Leptinotarsadecemlineata, and insecticidal RNAi in transgenic crops are reported to be near commercial release.

Despite these substantial advances, many RNAi studies on insects have yielded moderate or variable knock-down of gene expression, with limited effects on insect phenotype and performance. These problems apply particularly to plant sap-feeding insects, such as whiteflies, aphids, psyllids and planthoppers, including major pests and vectors of plant viruses, where RNAi against essential genes often reduces growth or reproduction, but has small or no effect on survivorship. A possible cause of the limited efficacy of in planta RNAi against many insects is that the plant RNAi molecules can be degraded by non-specific nucleases in the insect saliva, gut lumen or hemolymph. Accordingly, there is a need in the art for a method of increasing the efficacy of RNAi in plant sap-feeding insects.

### SUMMARY OF THE INVENTION

The present invention, in general, features methods and compositions for controlling plant infestations by repressing, delaying, silencing, or otherwise reducing gene expression within a particular pest.

In one aspect, the invention features a method including administering to a phloem sap-feeding insect one or more dsRNAs capable of suppressing activity of one or more RNAi-suppressing nuclease genes expressed in the gut of the insect and one or more dsRNAs capable of suppressing one or more osmoregulatory genes expressed by the insect. In some embodiments, the phloem sap-feeding insect is an aphid, a whitefly, a psyllid, a mealybug, a planthopper, or a leafhopper. In some preferred embodiments, the whitefly is Bemisiatabaci. In other preferred embodiments, the aphid is the pea aphid, Acyrthosiphonpisum.

In some embodiments of the previous aspect, the nuclease is a dsRNAse. In some embodiments, the osmoregulatory gene is an aquaporin or a glucohydrolase of family GH-13. In some embodiments in which the osmoregulatory gene is a glucohydrolase, the glucohydrolase is a sucrase. In some embodiments, the osmoregulatory gene is an aquaporin.

In preferred embodiments of the previous aspect, the method includes administration of two dsRNAs that suppress the activity of two dsRNAses, a dsRNA that suppresses an aquaporin, and a dsRNA that suppresses a sucrase.

In some embodiments of the previous aspect, the administration of dsRNAs is performed in planta. In yet other embodiments, the administration of dsRNA is performed in an artificial diet.

In another aspect, the invention features a plant that is resistant to a phloem sap-feeding insect, wherein the plant includes one or more dsRNAs capable of suppressing activity of one or more RNAi-suppressing nuclease genes expressed in the gut of the insect and one or more dsRNAs capable of suppressing one or more osmoregulatory genes expressed by the insect. In some embodiments, the insect that feeds on the plant is an aphid, a whitefly, a psyllid, a mealybug, a planthopper, or a leafhopper. In some preferred embodiments, the whitefly is Bemisiatabaci. In other preferred embodiments, the aphid is the pea aphid, Acyrthosiphonpisum.

In some embodiments of the previous aspect, the nuclease is a dsRNAse. In some embodiments, the osmoregulatory gene is an aquaporin or a glucohydrolase of family GH-13. In some embodiments in which the osmoregulatory gene is a glucohydrolase, the glucohydrolase is a sucrase. In some embodiments, the osmoregulatory gene is an aquaporin.

In preferred embodiments of the previous aspect, the plant includes two dsRNAs that suppress the activity of two dsRNAses, a dsRNA that suppresses an aquaporin, and a dsRNA that suppresses a sucrase.

In another aspect, the invention features a composition including one or more dsRNAs capable of suppressing activity of one or more RNAi-suppressing nuclease genes expressed in the gut of the insect and one or more dsRNAs capable of suppressing one or more osmoregulatory genes expressed by the insect.

Other features and advantages of the invention will be apparent from the following description of the preferred embodiments and from the claims.

### BRIEF DESCRIPTION OF DRAWINGS

FIGS. 1A to 1D depict Northern blot analyses of ds-GFP in transgenic tomato plants and whiteflies feeding on the plants. FIG. 1A depicts a map of transformation vectors for ds-GFP expression, designed to produce 370 bp hairpin-ds-GFP (hp-ds-GFP) under companion cell-specific promoter CmGAS or AtSUC2. FIG. 1B depicts 370 nt ds-GFP (top) and 21-25 nt small RNA (bottom) in the plants. FIG. 1C depicts 370 nt ds-GFP in cohorts of ca. 250 whiteflies that had fed on transgenic plants for two weeks. FIG. 1D depicts RNA gel blot analysis of total RNA extracted from the scion apex heterografted (GAS:4 and SUC2:11) plants. Numbers indicate transgenic tomato lines, with WT, the negative control, comprising tomato plants transformed with the empty vector. Twenty µg total leaf RNA or whitefly RNA was separated on 8% denatured urea-acrylamide gel, with the HMW RNA hybridization protocol for full-length ds-GFP detection and LMW RNA hybridization protocol for small RNA detection. The rRNA loading control is SybrGold stained 5S rRNA.

FIGS. 2A to 2E depict Northern blot analyses of ds-GFP in transgenic tomato plants and whiteflies feeding on the plants. FIG. 2A depicts plant RNA with HMW RNA extraction and HMW RNA hybridization protocols. FIG. 2B depicts plant RNA with LMW RNA extraction and LMW RNA hybridization protocols. FIG. 2C depicts whitefly with HMW RNA extraction and HMW RNA hybridization protocols. FIG. 2D depicts whitefly with LMW RNA extraction and LMW RNA hybridization protocols. FIG. 2E depicts RNA extracted from the scion apex heterografted (GAS:4 and SUC2:11) plants. RNA was extracted from tomato leaves (FIGS. 2A, 2B, 2E) and from ca. 250 adult whiteflies that had fed on transgenic plants for two weeks (FIGS. 2C, 2D), and the samples (20 µg RNA per lane) were separated on 8% denatured urea-acrylamide gel. Numbers indicate transgenic tomato lines, with Ev, the negative control, comprising tomato plants transformed with the empty vector. WT, wild-type tomato, MW, molecular weight. The rRNA loading control is SybrGold stained 5S rRNA. The bands of molecular weight intermediate between 370 nt GFP and candidate sRNAs (21-25 nt) were interpreted as non-specific binding of plant and whitefly RNA to the GFP probe because they were detected in both the experimental samples and Ev controls. The LMW RNA extraction protocol (FIGS. 2B, 2D) reduced, but did not exclude HMW RNA, particularly yielding multiple bands >> 21-25 bp in the plant samples (FIG. 2B) that were detectable with the low stringency of the LMW hybridization protocol required to visualize 21-25 bp bands.

FIGS. 3A to 3B depict Northern blot analyses of ds-GFP administered to whiteflies via artificial diet. FIG. 3A depicts a Northern blot of diet supplemented with 1 µg ds-GFP µl⁻¹ and harvested immediately (0 h) and after incubation for 72 h without whiteflies (-) or with whiteflies (+), with 100 ng RNA loaded per well. FIG. 3B depicts a Northern blot of whiteflies that had fed on two diets containing ds-GFP (+) or ds-GFP-free diets (-), with 20 µg total RNA loaded in each lane and Sybr-Gold stained rRNA as loading control. The LMW RNA hybridization protocol was used for both blots. For the best visibility, the 21-25 nt small RNA in whitefly was cut from the same blots with brightness adjustment.

FIGS. 4A to 4B depict the dsRNase genes of Bemisiatabaci. FIG. 4A depicts a neighbor-joining phylogenetic tree constructed using the protein sequence of the conserved DNA/RNA non-specific nuclease domain of insect dsRNase genes. The numbers at the branches indicate the %bootstrap support, based on the frequency of the clusters for 1000 bootstraps. The dsRNase sequences were: Bombyxmori 2 (NP_001091744.1), Papiliomachaon (XP_014355571.1), Spodopteralittorallis (CAR92522.1), Spodopterafrugiperda (CAR92521.1), Aedesaegypti (XP_001648469.1), Drosophilamelanogaster 1 (NM_140821.4), D.melanogaster 2 (NP_649078.1, CG3819), Triboliumcastaneum 1 (XP_973587.1), T.castaneum 2 (XP_970494.1), Schistocercagregaria 1 (KJ135008), S.gregaria 2 (KJ135009), S.gregaria 3 (KJ135010), S.gregaria 4 (KJ135011), Acyrthosiphonpisum (ACYPI008471), Myzuspersicae (MYZPE13164_0_v1.0_000125730.4_pep) and Bemisiatabaci 1(KX390872), B.tabaci 2 (KX390873) and B.tabaci 3 (Unigene11878_BT_Q_SG_ZJU). FIG. 4B depicts a qRT-PCR analysis of the expression of BtdsRNase-1 (top) and BtdsRNase-2 (bottom) in dissected guts of B.tabaci, relative to the whole body (Wb). Mean ± s.e. from 3 replicates were showed.

FIGS. 5A to 5D depict the effect of RNAi against dsRNase on the abundance of ds-GFP administered orally to whiteflies. FIG. 5A depicts experimental design. FIG. 5B depicts Northern blots of ds-GFP fragment following treatment with ds-dsRNase1. FIG. 5C depicts Northern blots of ds-GFP fragment following treatment with ds-dsRNase2. FIG. 5D depicts Northern blots of ds-GFP fragment following treatment with ds-dsRNase1&2, with the full-length 370 nt GFP and ca. 21 nt regions taken from the same blot, and SybrGold-stained 5S rRNA as loading control. The LMW RNA hybridization protocol was used for all blots. Relative density of the 370 nt ds-GFP bands on the blots (mean ± s.e), with ANOVA results and significantly different treatments by post hoc test indicated by different letters (right histogram graph). Hybridization of the GFP probe with 10 ng chemically-synthesized ds-GFP was used for normalization across the different blots (as showed in probe lane). The exposure time for each blot was optimized independently, to ensure that none of the bands on each blot was saturated.

FIGS. 6A to 6B depict whitefly dsRNase genes’ relative expression level with administration of ds-dsRNase 1, ds-dsRNase 2 or ds-dsRNase 1&2, either synchronously with ds-GFP or 3 days prior to adding ds-GFP. FIG. 6A depicts the relative expression level of dsRNase 1. FIG. 6B depicts the relative expression level of dsRNase 2. Gene expression was normalized to RPL13, and mean ± s.e. from 3 replicates were showed. Different letters indicate significantly different treatments by post hoc test following one-way ANOVA.

FIG. 7 depicts northern blots of ds-GFP fragment following treatment with ds-dsRNase1, ds-dsRNase2 and ds-dsRNase1&2, with SybrGold-stained 5S rRNA as loading control. The LMW RNA hybridization protocol was used for all blots. The exposure time for each blot was optimized independently, to ensure that none of the bands on each blot was saturated and so ensure the reliability of the data in FIG. 5B. Biological variability and the criteria for optimizing exposure time (above) contribute to the among-experiment variation. No: no treatment; Syn: synchronous treatment with ds-GFP and ds-RNase; Pre: pretreatment with ds-dsRNase (see FIG. 5A for details).

FIGS. 8A to 8B depict expression of dsRNase genes in the whole body and dissected gut adult whiteflies reared for 6 days on artificial diets. Gene expression was normalized to RPL13. Mean ± s. e. from 3 replicates are shown. * indicate significantly different by Student’s t-test.

FIGS. 9A to 9C depict RNAi against osmoregulation genes in adult B.tabaci on artificial diet. dsRNA (100 ng µl⁻¹ diet) were ds-GFP, ds-dsRNase1&2, and dsRNA against osmoregulation genes AQP1 and SUC1 with (+) or without (-) ds-dsRNase1&2 administered over 6 days. FIGS. 9A and 9B depict qRT-PCR analysis of AQP1 and SUC1 expression, respectively, relative to dsRNA-free diet, normalized to RPL13. Mean + s.e, 3 reps. FIG. 9C depicts the number of dead insects (mean + s.e. for 10 replicates of 40 insects) at day-6. Different letters indicate significantly different treatments by Fisher’s LSD test.

FIG. 10 depicts relative expression level of whitefly SUC1, AQP1 and dsRNase 1&2 genes in adult whiteflies reared for 6 days on artificial diets supplemented with ds-dsRNase1&2 (+), with ds-dsRNase1&2-free diets (-) as control. FIG. 10 depicts the relative expression level of dsRNasel. FIG. 10 depicts the relative expression level of dsRNase2. Gene expression was normalized to RPL13, and mean ± s. e. from 3 replicates are shown. Different letters indicate significantly different treatments by post hoc test following one-way ANOVA.

FIG. 11 depicts in planta RNAi against osmoregulation and dsRNase genes in adult B.tabaci.. The number of dead insects at the end of the 8-day experiment in the duplicate cages on each plant was pooled. Different letters indicate significantly different treatments by Fisher’s LSD test.

### DETAILED DESCRIPTION

In general, the present technology provides a system/method for the use of RNAi suppression of RNAi suppressors. This dramatically increases the efficacy of RNAi against essential genes, so achieving high mortality of an insect pest. Our methods described herein also provide in planta RNAi against sucking insect pests of agriculture. The technology can be stacked with transgenes (e.g. Bacillusthuringiensis [Bt]) that are effective against chewing pests (beetles, caterpillars etc.).

It is demonstrated herein that the dsRNA (the RNAi molecule) is degraded nonspecifically in the gut of the whitefly Bemisiatabaci and the pea aphid Acyrthosiphonpisum, and we identify gut nucleases in both the whitefly and aphid that are suppressors of RNAi. We also show both reduced dsRNA degradation and increased efficacy of RNAi against osmoregulation genes in whiteflies that are co-administered dsRNA against the nucleases and osmoregulation genes.

The purpose of our study was to identify the factors in the plant and insect that limit the efficacy of RNAi against phloem-feeding insects, and to use this information for improved design of RNAi. Our experiments were conducted on the whitefly Bemisiatabaci, which is a globally-important pest of many crops.

We hypothesized that whitefly RNAi may be limited by processing of dsRNA to small RNAs (sRNAs) in the plant and, additionally or alternatively, by nonspecific degradation of RNAi molecules in the insect. We tested these hypotheses by following the fate of dsRNA constructed against a 370 nt fragment of the green fluorescent protein (GFP) gene of the jellyfish Aequoreavictoria, administered to the insects via artificial diets and plant transgenesis. These experiments led us to identify non-specific degradation of dsRNA by B.tabaci, which we then reduced by RNAi against two candidate B.tabaci nuclease genes. In our final experiments, we tested the efficacy of stacking RNAi against the nuclease genes with RNAi against candidate essential genes of B.tabaci. Our genes of choice were an aquaporin and a glucohydrolase of family GH-13, which are candidate osmoregulation genes that protect the insect from rapid dehydration and death. We call these genes BtAQP1 and BtSUC1, respectively. Our results provide new insights into the fate of RNAi molecules administered to phloem-feeding insects by in planta RNAi, and the value of this technology as a novel insect pest control strategy.

Furthermore, as described herein, we have obtained information about phloem-mobile RNA molecules from the RNA content of both wild-type tissue grafted onto transgenic plants and phloem-feeding insects. When these two methods were applied in this study to stable transgenic tomato containing ds-GFP under two alternative phloem-specific promoters, they yielded the full-length 370 nt dsRNA, but not sRNA in the 20-25 nt range. This result cannot be attributed to technical difficulties in detecting sRNA because both full-length dsRNA and sRNA were detected in bulk transgenic leaf samples of the same total RNA content.

Further insight into the delivery of in planta RNAi to phloem-feeding insects was obtained from our comparison of the ds-GFP products in whiteflies feeding from ds-GFP transgenic plants and ds-GFP-supplemented artificial diet. Specifically, sRNA was detected in insects fed on ds-GFP via the diet but not the plant. These data evidence that non-specific nuclease activity in the insect gut degraded the dsRNA in the phloem sap, quantitatively preventing dsRNA delivery to gut cells, where siRNA is generated by the cytoplasmic RNAi machinery; but that the high concentrations of dsRNA in the artificial diets saturated the gut nuclease activity, such that a proportion of the ingested dsRNA was translocated to gut cells, yielding detectable sRNA in the insect.

As is shown herein, the application of RNAi in whiteflies to test whether RNAi-mediated suppression of dsRNase genes resulted in enhanced efficacy of RNAi against other insect genes, specifically the two predicted osmoregulation genes, AQP1 and SUC1. As predicted, orally-delivered ds-dsRNase1&2 both protected ds-GFP from non-specific degradation and increased the efficacy of RNAi against osmoregulation genes, as quantified by gene expression and survivorship of insects administered RNAi via artificial diet. These data demonstrate that the efficacy of RNAi in various insects may be significantly improved by RNAi-mediated suppression of dsRNase genes, and potentially other RNAi suppressors.

As is shown herein, our results provide proof of principle that the efficacy of RNAi against the whitefly B.tabaci can be enhanced by the dual strategies of, first, stacking RNAi against multiple genes with related physiological roles but distinct molecular functions and, second, using RNAi to suppress suppressors of RNAi. These approaches create wider opportunities for in planta RNAi as a control strategy against B.tabaci, which is a globally important crop pest, and offer a template for comparable strategies against other insect pests with poor or variable RNAi efficacy.

The invention is further described in the following examples, which do not limit the scope of the invention described in the claims.

### EXAMPLES

### Plants and Insects

Tomato plants ( Solanumlycopersicum cv. Florida Lanai) were grown in compost supplemented with Miracle-Gro® Water Soluble All Purpose Plant Food in climate-controlled chambers at 25±2° C. with a 14L:10D light cycle at 400 µmol/m²/s PAR.

The Bemisiatabaci MEAM1 culture (mtCO1 GenBank accession no. KM507785) was derived from a collection from poinsettia ( Euphorbiapulcherrima Willd. Ex Klotzsch) in Ithaca, NY, USA in 1989. The insects were maintained on 5-6-week-old tomato plants at 25±2° C. with a 14L:10D light cycle at 400 µmol/m²/s PAR. One-day-old adult males and female insects were caged to test plant leaves using BugDorm-1 Insect Cages (Bio Quip, Rancho Dominguez, CA) or custom clip-cages, and fed on a sterile artificial liquid diet containing 0.5 M sucrose and 0.15 M amino acids in Parafilm sachets. Some experiments used isolated guts, dissected with fine pins from adult female and male insects into phosphate-buffered saline.

### RNA Extractions

For total RNA isolation, leaves or whiteflies were homogenized in TRIzol® Reagent (Cat# 15596-026, Thermo Fisher Scientific, Waltham, USA) with 1:1 (vol) Lysing Matrix D Bulk beads (Cat# 116540434, MP Biomedicals, Santa Ana, USA) on a MP FastPrep-24™ homogenizer (Cat# 116004500, MP Biomedicals, Santa Ana, USA) with 5.5 M/S for 2 × 30 seconds. The lysed samples were centrifuged at 13,000 rpm for 3 min to remove the beads and RNA was isolated from the supernatant following the manufacturer’s instructions. RNA samples used for qRT-PCR were further processed to remove genomic DNA by incubation with 10 µl DNase 1, using the reagents and protocol in the RNase-free DNase set (Cat# 79254, Qiagen, Valencia, USA). To collect dsRNA from diets, samples of the liquid diet were combined with isopropanol (1:1, by vol) and 3 M sodium acetate pH 5.2 (1:10 by volume). The concentration of RNA was determined spectrophotometrically with a Nanodrop-2000 (Thermo Fisher Scientific, Waltham, USA), and RNA integrity was verified by denaturing formaldehyde gel-electrophoresis.

### Synthesis of cDNA and dsRNA

cDNA libraries for amplification of whitefly genes were prepared with oligo (dT) primers using the Reverse Transcription System (Cat# A3500, Promega, Madison, USA) following the manufacturer’s instructions. Candidate osmoregulatory genes, comprising aquaporin AQP1 (NCBI Accession KX390870), sucrase SUC1 (also known as α-glucohydrolase GH13-1) (NCBI Accession KX390871), and candidate nuclease genes dsRNasel (NCBI Accession KX390872) and dsRNase2 (NCBI Accession KX390873) identified in this study, were amplified from the cDNA template in a reaction mix containing 0.4 µM primers (Table 1), 2 U Invitrogen Platinum Taq DNA polymerase (Cat# 10966018, Thermo Fisher Scientific, Waltham, USA), 1.5 mM MgCl₂ and 200 ng cDNA template in 25 µl volume, using a Techne thermal cycler. The thermal profile comprised 2 min at 94° C. for initial denaturation, 30 cycles with 95° C. for 30 sec, 55° C. for 30 sec, 72° C. for 1 min (depending on products length, 1 kb/min) and final extension cycle of 72° C. for 5 min. Amplicon sequences were verified by Sanger sequencing, then introduced into PGEM-T vector (Cat# A1360, Promega, Madison, USA) and transformed into DH5α™ competent cells. The plasmid was extracted and, following confirmation by sequencing, used as template for dsRNA synthesis with primers listed in Table 1.

TABLE 1

| | | |
|---|---|---|
| Gene | Forward primer 5′-3′ | Reverse primer 5′-3′ |
| (a) primers for gene verification | | |
| dsRNase1 | CGCTGATGAAACCGGAAATG (SEQ ID NO: 1) | GCTTGTGGCACTCTTGTTATG (SEQ ID NO: 2) |
| dsRNase2 | CGTTGGCGCAGTTTGTAAAG (SEQ ID NO: 3) | CCACTCGCATTTGAGAGGAA (SEQ ID NO: 4) |
| SUC1 | GATCTAGCTTGGTGGGAGAAAG (SEQ ID NO: 5) | TGTATGTCGGGTGGTCTTTG (SEQ ID NO: 6) |
| AQP1 | CATAGTTGCTGAGTTCGTAGGG (SEQ ID NO: 7) | GCTTTGAAAGTGATGGCGTAAA (SEQ ID NO: 8) |
| (b) primers for dsRNA synthesis (with T7 promoter sequence: TAATACGACTCACTATAGG (SEQ ID NO: 9)) | | |
| dsGFP | ATGGGTAAAGGAGAAGAAC (SEQ ID NO: 10) | ATCCTGTTGACGAGGGTGTC (SEQ ID NO: 11) |
| dsRNase1 | TACGAACATTAACGGGAACGACGG (SEQ ID NO: 12) | GGTATAGCTGGGTTCACCTTCAGTG (SEQ ID NO: 13) |
| dsRNase2 | CAGTGGCAAATCATCAATGCG (SEQ ID NO: 14) | ATGTGGAGATTTATTTACAGCCAG (SEQ ID NO: 15) |
| SUC1 | GATCTAGCTTGGTGGGAGAAAGG (SEQ ID NO: 16) | GGGGAGCGAAAAATCGGAGA (SEQ ID NO: 17) |
| AQP1 | TTCGTAGGGACTTTGCTGTTAG (SEQ ID NO: 18) | CGGATTGATGTGACAACCACTAA (SEQ ID NO: 19) |
| (c) primers for qRT-PCR | | |
| dsRNase1 | GAAACTCGCTCCTCTTGTAGTT (SEQ ID NO: 20) | TGTCTGCTCTTCCTGTCTTATTC (SEQ ID NO: 21) |
| dsRNase2 | CGAGTGCACGAGTAGTGTAAA (SEQ ID NO: 22) | CACACCCACATCAGAGGTAAA (SEQ ID NO: 23) |
| SUC1 | GACTGGTATGCTGCTCTCCC (SEQ ID NO: 24) | CATCTGAAGCATGTGCAGCC (SEQ ID NO: 25) |
| AQP1 | GGAGCCATCTGTGGAGCAAT (SEQ ID NO: 26) | AGTGCTTCTATCGCCACACC (SEQ ID NO: 27) |
| RPL13 | TAAATTCGACTCGACTCACGGT (SEQ ID NO: 28) | CTCCACCACATACTCGGCTC (SEQ ID NO: 29) |
| (d) Primers for identification of positive transformants | | |
| GFP | ATGGGTAAAGGAGAAGAAC (SEQ ID NO: 30) | ATCCTGTTGACGAGGGTGTC (SEQ ID NO: 31) |

The dsRNA was synthesized using the AmpliScribe TM T7-Flash Transcription Kit (Cat# ASF3257, Epicentre Biotechnologies, Madison, USA), according to the manufacturer’s instructions. The templates were the EGFP-pBAD plasmid (plasmid #54762, AddGene Plasmid Repository) for ds-GFP, and plasmids obtained for the whitefly genes identified above. The dsRNA product was quantified by Nanodrop, and run on a gel with 1 kb plus molecular weight ladder (Cat# 10787026, Thermo Fisher Scientific, Waltham, USA) to confirm the predicted size.

### Construction of dsRNA Expression Cassette

The dsRNA expression cassette was constructed in the pHANNIBAL vector. A 370 nt GFP sequence was amplified and inserted in inverted orientation into pHANNIBAL using the PDK intron as a spacer and different restriction sites, XhoI and EcoRI for sense GFP and HindIII and XbaI for the inverted GFP sequence. Two phloem-companion cell-specific promoters, Galactinol Synthase from melon Cucumismelo (CmGAS) and sucrose-H+ symporter from Arabidopsisthaliana (AtSUC2) were cloned and inserted into the binary vector pER8 using XhoI/SpeI and XhoI restriction sites separately. The GFP-intron-rGFP cassette was assembled and inserted downstream of the promoter in pER8 vector using a XhoI restriction site for AtSUC2 and SpeI site for CmGAS 1. For RNAi constructs with multiple targets, ca. 150 bp of the different gene fragments were, first, fused together through Gibson assembly method into XhoI and EcoRI sites in pHANNIBAL vector, or reversed fused sequence into HindIII and XbaI sites (e.g. XhoI-RNase1+RNase2+AQP1-EcoRI for forward sequence cloning and HindIII-AQP1+ RNase2+ RNase1-XbaI for inverted orientation cloning). The final dsRNA expression cassettes were assembled downstream of AtSUC2 promoter in pER8 at Xho I site. All vectors were verified by sequencing.

### Generation of Tomato Transgenics with dsRNA Gene Construct

The binary vector pER8 was introduced via electroporation into Agrobacteriumtumefaciens strain LBA4404. Kanamycin selection at 500 µg/ml was used to select for transformants. Transformation of the tomato plants was performed. Putative transgenic plants were transferred to soil and maintained in an incubator at 25±2° C. with a relative humidity of 60 - 70% and with a 14L:10D light cycle at 400 µmol/m²/s PAR. Total DNA was extracted from leaves of 5-6-week-old transgenic plants and verified for transformation by PCR with sequence specific primers (Table 1). Eight transgenic lines were confirmed for each promoter.

### Administration of dsRNA to Whiteflies

dsRNA was administered to adult insects either via chemically-defined diets (at 0.1-1 µg/µl, varying with experiment) or via transgenic tomato lines. For analysis of the fate of ds-GFP, 100 whiteflies were administered to each diet cage and ca. 250 whiteflies were caged to each plant. For insect performance experiments on artificial diets, 10 replicate groups of 40 adult whiteflies (one day post-emergence) were applied to each diet treatment and mortality was monitored daily over 6 days, with insects transferred to fresh diet containing dsRNA every two days. At the end of each experiment, all live insects were transferred to 500 µl TRIzol® Reagent (Cat# 15596-026, Thermo Fisher Scientific, Waltham, USA) and stored at -80° C. prior to isolation of total RNA (as above). Transgenic plants at the 4-5 leaf stage were used for performance assays of insects on plants. Ten 2-day-old adult insects (1:1 sex ratio) were transferred in a clip-cage to the abaxial surface of each of the second and third leaf of each plant, with 4 replicate plants for all treatments, apart from the ds-dsRNase plants with three replicates. Eight days after infestation, the number of dead insects was scored, and surviving insects were flash-frozen and stored at -80° C. for RNA extraction.

### Northern Blots

RNA extracted from whiteflies and plants was separated on denatured polyacrylamide-urea gels (SequaGel - UreaGel System, cat# EC-833, National Diagnostics, Atlanta, GA, USA) containing 8% monomers. The gel was pre-run at 250 V in 0.5 X TBE buffer for 30 min, then 20 µg sample RNA was combined with an equal volume of Gel Loading Buffer II (Cat# AM8547, Thermo Fisher Scientific, Waltham, USA) and heated at 95° C. for 4 minutes to denature the RNA. Samples were loaded in urea-cleaned wells and run at 250 V in 0.5 × TBE buffer until the loading dye migrated to the far end of the gel. Uniform sample loading was confirmed by staining of 5S rRNA with SYBR Gold, followed by transfer to Hybond-NX membrane (Cat# RPN 203T, GE Healthcare, Wilkes-Barre, PA) with the Owl™ HEP Series Semidry Electroblotting Systems at 0.4 A for 1 hour. The transferred RNA was cross-linked using a UV crosslinker at 120 kJ for 30 sec for HMW RNA and for LMW RNA.

The GFP probe used for northern blotting was generated using the MAXIscript® In Vitro Transcription Kit (Cat# AM1308, Thermo Fisher Scientific, Waltham, USA) with 3.125 µM alpha-³²P UTP (10 mCi/ml, 800 Ci/mmol, Perkin Elmer, Waltham, USA), 5 µM UTP and 100 µM ADP. Unincorporated ³²P-label was removed using a RNeasy mini column (Cat# 74104, Qiagen, Venlo, Limburg, USA). The ³²P-labeled probe was brought to 100 µl with nuclease-free water, mixed with 350 µl buffer RLT (RNeasy mini kit, Cat# 74104, Qiagen, Venlo, Limburg, USA) and 250 µl 100% ethanol, collected onto a RNeasy mini column, washed twice with buffer RPE (from RNeasy mini kit, Cat# 74104, Qiagen, Venlo, Limburg, USA), and eluted with 40 µl nuclease-free water. For each membrane, half of the probe was heated at 95° C. for 4 min and immediately added to the pre-hybridized membrane at 2 × 106 cpm/ml final concentration. For HMW RNA detection, pre-hybridization and hybridization used Ambion Ultrahyb buffer (CAT# AM8670, Thermo Fisher Scientific, Waltham, USA) according to the manufacturer’s protocol. The blot was washed twice in 2× SSC, 0.1% SDS buffer at 68° C., followed by two washes in 0.1× SSC, 0.1% SDS buffer at the same temperature. For LMW RNA detection, the blot was pre-hybridized in hybridization buffer (5 × SSC, 20 mM Na2HPO4 (pH 7.2), 7% SDS, 2 × Derhardt’s solution) at 50° C. for at least two hours, then hybridized with a final concentration of 2.5 × 106 cpm/ml probe in the same buffer at 50° C. overnight. The membrane was washed four times in non-stringent wash buffer (3× SSC, 25 mM NaH2PO4 pH 7.5, 5% SDS) and once in stringent wash buffer (1× SSC, 0.1% SDS), and then exposed for autoradiography. The signal was collected on phosphor screen (Molecular Dynamics) and scanned using a Typhoon 9400 fluorescent imager. ImageJ was used for ds-GFP band density analyses.

### qRT-PCR

To quantify the expression of target whitefly genes, qRT-PCR was performed with RNA extracted from three biological replicates of whiteflies. cDNA was prepared using random primers of High-Capacity cDNA Reverse Transcription Kit or SuperScript™ II Reverse Transcriptase (Cat# 4368814 and 18064014, Thermo Fisher Scientific, Waltham, USA) following the manufacturer’s instructions. For qRT-PCR, the 20 µl reaction mix comprised 10 µl Master Mix (Bio-Rad, Hercules, CA) or Power SYBR Green PCR Master Mix (Applied Biosystems, Carlberg, CA, USA)], precisely 1 µl cDNA template and 0.5-2 µl 10 µM primers qRT-PCR primers (Table 1) designed with Primer Premier 5.0 software (Premier Biosoft International, Palo Alto, CA). Amplifications were conducted in a C1000TM Thermal cycler (Bio-Rad, Hercules, CA) with the following thermal profile: 95° C. for 5 min, 40 amplification cycles of 95° C. for 15 sec, 55° C. for 30 sec, and dissociation cycle of 95° C. for 15 sec, 55° C. for 15 sec then brought back to 95° C. Dissociation curves confirmed single peaks of the predicted size without primer dimerization. All assays included three technical replicates with template-free and non-RT as controls; and the relative expression was calculated using the 2⁻^{ΔΔ}^{Ct} method, normalized to the whitefly 60S ribosomal protein L13a (RPL13) gene. Mean Ct value of three technical replicates was calculated per sample.

### Identification and Phylogenetic Analysis of Candidate Whitefly dsRNase Genes

To obtain an initial set of candidate nucleases in B.tabaci, the translated sequence of the non-specific nuclease Bombyxmori, which has been demonstrated to degrade dsRNA and suppress RNAi, was BLASTed (E value < 1.0 e-10) against the translated RefSeq genes in the B.tabaci genome. The resultant B.tabaci genes were analyzed by Signalp and NCBI conserved domain database for signal peptide and conserved domain. For analysis of gene phylogenies, a neighboring-joining tree was constructed of the conserved DNA/RNA non-specific nuclease domain amino acid sequences (MEGA 6.06).

### Tomato Plant Grafting

For each graft, a scion with few mature leaves from 5-6-week-old wild-type tomato was cut and inserted to a transgenic RNAi plant of the same age. The graft site was fastened with Parafilm; and the plant was covered with a plastic bag and kept in darkness at 25° C. for 48 h. Lighting was gradually increased over the next 3-4 days, and the bag was removed at day-7. Wildtype scions were grafted to two copies per GAS:ds-GFP line for four plants and copies of the two SUC2:ds-GFP lines for 11 plants. The plants were harvested 3 weeks after grafting for RNA isolation and northern blotting analysis of ds-GFP fragments.

### Statistical Analysis

All data sets conformed to the expectations of normality by the Anderson Darling test and homogeneity of variance by the Levine and Bartlett tests. They were analyzed by one-way ANOVA with Fisher’s LSD post hoc test. Statistical analyses were conducted with JMP software (SAS Institute, Miami, USA) and Minitab 17.

### Delivery of Plant dsRNA to Whiteflies

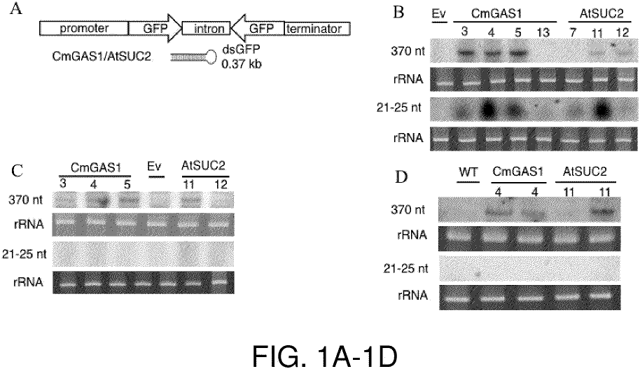

The first experiments investigated the fate of dsRNA expressed under companion cell-specific promoters CmGAS from melon, Cucumismelo (expressed in companion cells of minor veins of the leaf) and AtSUC2 from Arabidopsisthaliana (expressed in all companion cells) in transgenic lines. The 370 bp ds-GFP construct (FIG. 1A) used in these experiments had no sequence homology to tomato or whitefly genes, to ensure that processing of the dsRNA would not be altered by sequence-specific effects of the dsRNA on gene expression in the plant or insect.

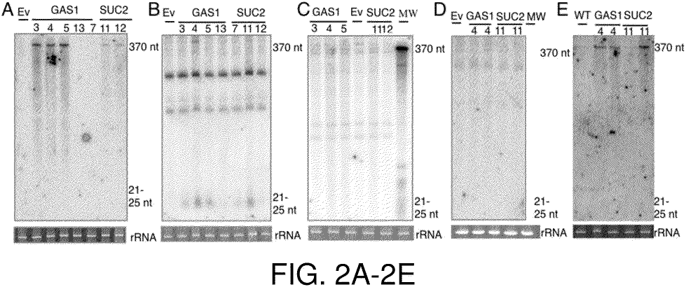

Northern blotting of ds-GFP fragments expressed under the CmGAS and AtSUC2 promoters in 5-6-week-old transgenic tomato lines yielded a band at ca. 20-25 nt in all lines and at 370 nt in most lines (FIG. 1B). These results are compatible with the interpretation that plant Dicer enzyme(s) cleaved the full-length ds-GFP into sRNA(s). (Our methods could not discriminate whether the sRNA product was one or multiple molecules in the range 20-25 nt.) Smearing of the signal down the gel, indicative of non-specific degradation of ds-GFP, was very limited in northern blots of plant RNA (FIG. 2A, whole gel picture of FIG. 1B). A 370 nt band hybridizing to the GFP probe was also detected in whiteflies that had been reared on the transgenic plants (FIG. 1C). The blots for the whiteflies bore a strong smeared signal over an extended range of the gel, indicative of non-specific degradation, and no detectable sRNA signal. (FIGS. 2C&D, whole gel picture of FIG. 1C).

We postulated two alternative explanations for the apparent absence of GFP sRNA in the whiteflies: that the sRNA, first, is not phloem-mobile and consequently not ingested by the whiteflies; and, second, is ingested but degraded in the whitefly. To test for the phloem mobility of the full-length and sRNA, control scions were grafted onto transgenic plants expressing ds-GFP under CmGAS or AtSUC2 promoters, with homografts onto wild-type (WT) plants as negative control. Three weeks after grafting, entire scion apices were excised and processed for GFP-RNA by northern blotting. The 370-nt band, but not the sRNA band, was detected (FIG. 1D). These data indicate that the processed sRNA derived from the ds-GFP is not phloem-mobile, and therefore not available to the whiteflies feeding on phloem sap from the transgenic plants.

### dsRNA Degradation in the Whiteflies

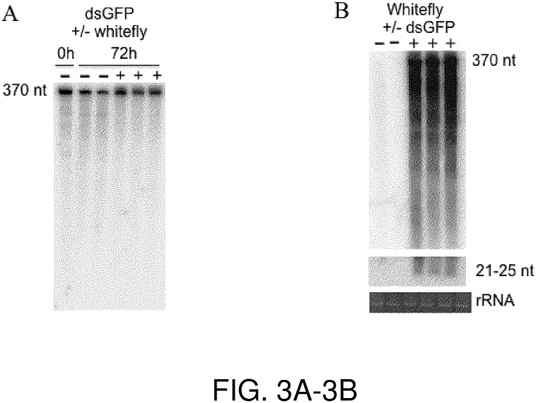

To investigate the fate of RNAi-related molecules in the whiteflies further, adult insects were administered ds-GFP via artificial diet. The 370 nt ds-GFP was recovered in northern blots of both the diet on which the whiteflies had fed and diet without whiteflies (FIG. 3A). Parallel analysis of the whiteflies yielded the 370 nt GFP band (as in the diet), a smear of signal along the length of the gel, indicative of non-specific degradation, and a faint sRNA band at ca. 21 nt (FIG. 3B). These data suggest that extra-oral degradation of dsRNA, as mediated by salivary secretions in Lygus bugs, is not substantial in the whitefly, but that an appreciable portion of ds-GFP ingested by the whiteflies is subjected to non-specific degradation within the insect body. We hypothesized that dsRNA ingested by whiteflies is subjected to non-specific degradation in the gut lumen, restricting the availability of dsRNA molecules for uptake by cells of the gut epithelium and intracellular processing by the RNAi machinery. To test this hypothesis, we applied phylogenetic methods to identify candidate nuclease genes in the B.tabaci genome.

### Phylogenetic Analysis of Candidate dsRNase Genes in the Bemisia Tabaci Genome

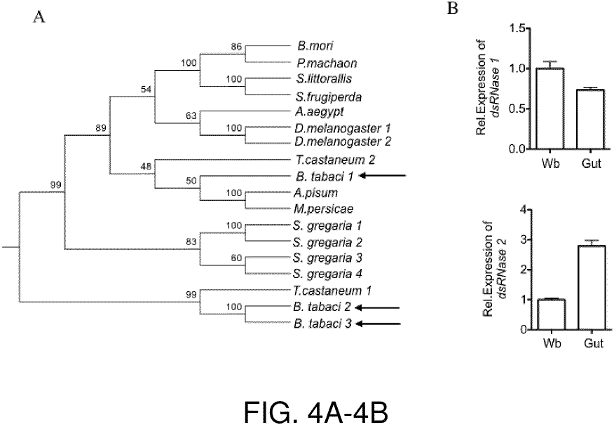

Our strategy to identify candidate nuclease(s) in the B.tabaci genome was to identify orthologs of a Bombyxmori DNA/RNA non-specific nuclease gene (BmdsRNase) that has been validated experimentally to cleave dsRNA and reduce the efficacy of RNAi. The B.mori dsRNase was BLASTed (E value < 1.0 e-10) against transcriptome databases of the whole body, salivary gland and gut of B.tabaci, yielding three B.tabaci sequences (dsRNasel, dsRNase2, dsRNase3 ) with a single DNA/RNA non- specific nuclease domain (NCBI conserved domain database) and a predicted signal peptide (SignalP). A phylogenetic tree constructed by the neighbor-joining method using the amino acid sequence of the conserved DNA/RNA non-specific nuclease domain from multiple insect species aligned the B.tabaci dsRNasel with aphid nucleases with moderate bootstrap support, and dsRNase2 and dsRNase3 with nuclease-1 of Triboliumcastaneum, with excellent bootstrap support (FIG. 4A).

We amplified part of the predicted full-length cDNA sequences of dsRNasel and dsRNase2 from whole body and gut cDNA libraries of adult B.tabaci, but failed to amplify dsRNase3. Complementary searches of the B.tabaci whole body, gut and salivary gland transcriptome databases yielded a few dsRNase3 reads only in the salivary gland transcriptome, suggesting that this gene is weakly expressed in the adult insects. Validating these transcriptome data, qRT-PCR analysis of the B.tabaci used in this study confirmed that dsRNasel and dsRNase2 are expressed, with three-fold enrichment of dsRNase2 expression in the gut relative to the whole body (FIG. 4B).

### Effect of RNAi Against dsRNase Genes on ds-GFP Ingested by the Whiteflies

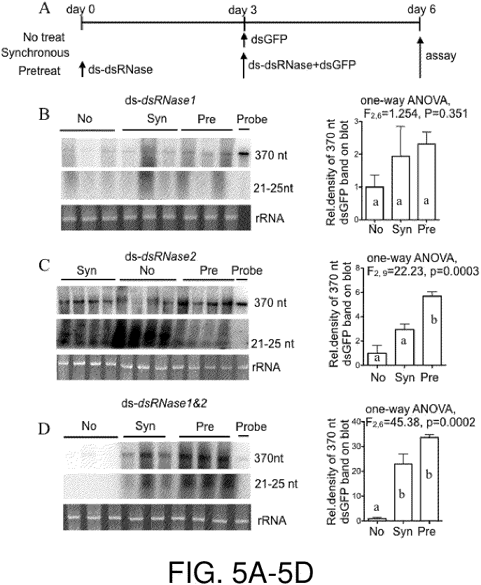

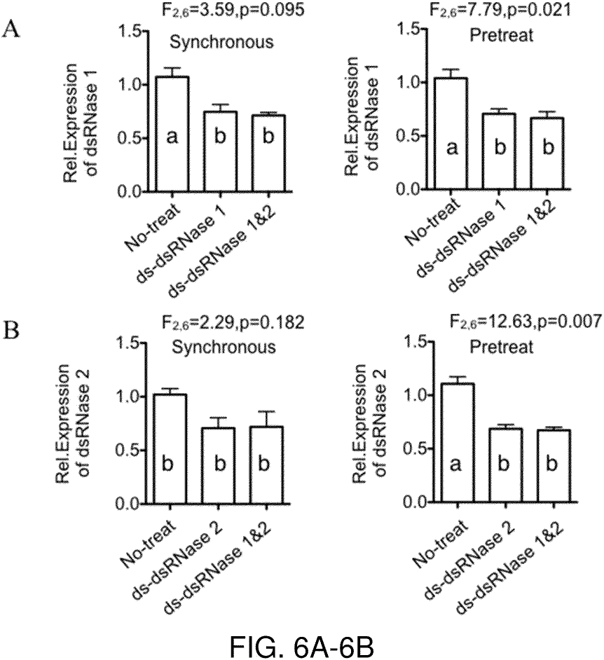

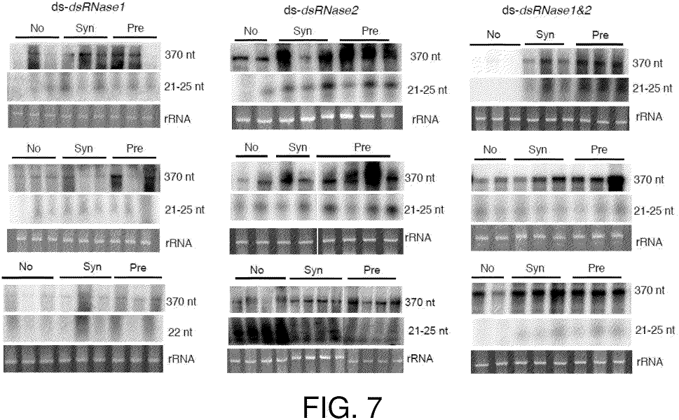

We next asked whether inhibiting whitefly dsRNase genes could protect ds-GFP from nonspecific degradation and thus improve RNAi. Chemically synthesized dsRNA against dsRNasel or dsRNase2 was fed to the whiteflies either synchronously or 3 days prior to adding ds-GFP, with dsRNase-free treatment as the control (FIG. 5A). qRT-PCR showed that administration of dsRNA against dsRNasel and dsRNase2 reduced their expression by 25-30% (FIG. 6). Northern blots revealed that the intensity of the full-length ds-GFP band was elevated in whiteflies administered RNAi against the RNase genes (FIG. 7), and this effect was significant for the whiteflies pretreated with dsRNA against dsRNase2 and both dsRNases, but not dsRNase1 (FIGS. 5B-D). The signal for the ca. 21 nt GFP was also more robust in the treatments that included ds-RNase2 (FIGS. 5B-D).

### Effect of ds-dsRNase on Efficacy of RNAi Against Whitefly Osmoregulation Genes

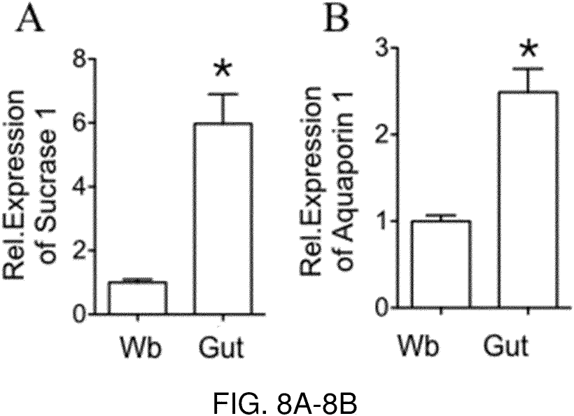

The demonstration (FIG. 5) that RNAi against the whitefly dsRNase genes is protective for ds-GFP provided the basis to investigate whether the efficacy of RNAi against whitefly genes of interest can be improved by combination with RNAi against the dsRNase genes. Our experiments focused on whitefly genes AQP1 and SUC1, with the predicted function of protecting the insect against osmotic dysfunction. Because these genes have been identified principally by bioinformatics methods, we first conducted qRT-PCR experiments that confirmed the expression of these genes, including their enriched expression in the gut (by 6-fold for SUC1 and 2.3-fold for AQP1), in the adult whiteflies used for our experiments (FIG. 8).

In the first RNAi experiment, the dsRNAs were delivered to adult whiteflies via artificial diet over a time-course of 6 days. The experimental treatments were dsRNA against the whitefly AQP1 and SUC1, either separately or in combination, and with or without the dsRNA against RNase1 and RNase2. The control samples comprised diets with ds-GFP or ds-dsRNase1&2, and dsRNA-free diets.

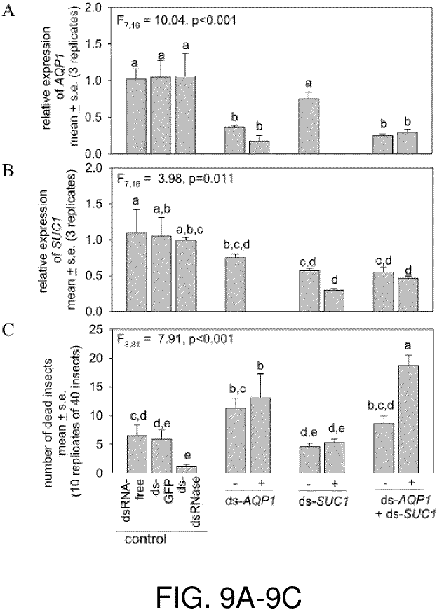

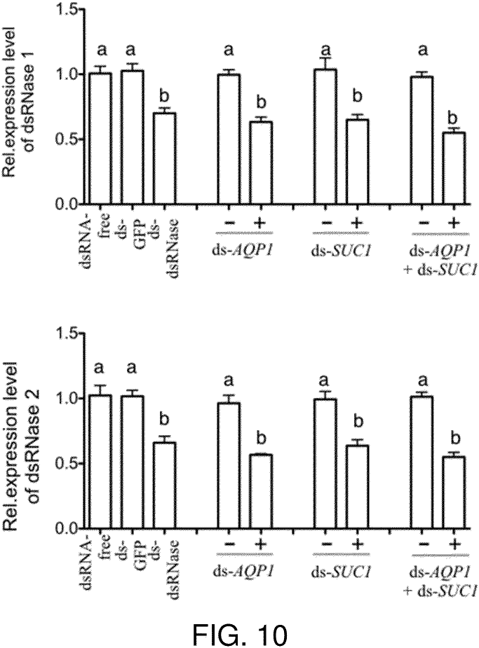

The impact of the dsRNAs administered via the artificial diet on the performance of the whiteflies was quantified as mortality of the insects over the 6-day test period. Relative to the control diets (ds-GFP and dsRNA-free), mortality was significantly elevated only in the two treatments containing both ds-AQP1 and ds-dsRNase1&2. In the absence of ds-AQP1, ds-SUC1 had no discernible effect on whitefly mortality. However, ds-SUC1 functioned synergistically with ds-AQP1 in the presence of ds-dsRNase1&2 to yield mortality approaching 50% and significantly greater than all other treatments. Surviving insects on day-6 were used for gene expression analysis by qRT-PCR. The ds-dsRNase treatments reduced expression of the cognate dsRNase genes by 30-35% (FIG. 10) but, when administered as the sole dsRNA, did not affect expression of the target osmoregulation genes (FIGS. 9A & B). The effect of the ds-dsRNase on gene expression differed between AQP1 and SUC1. AQP1 expression was significantly reduced by 64-83% in insects in all treatments that included ds-AQP1, with no significant additional effect of co-administration of either ds-SUC1 or ds-dsRNase (FIG. 9A); and SUC1 expression was significantly reduced (by 54-70%) only in treatments that included both ds-SUC1 and ds-dsRNase (FIG. 9B). In other words, the efficacy of suppressing expression of the whitefly dsRNase genes as a tool to enhance RNAi varies across different whitefly genes.

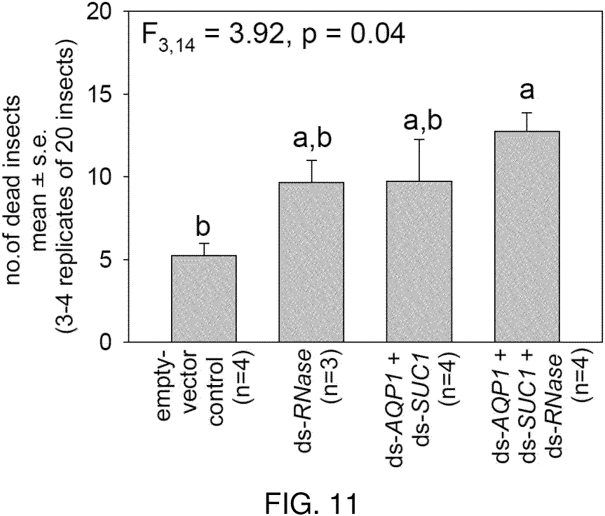

We then investigated the response of the whiteflies to RNAi against the osmoregulation genes administered in planta (FIG. 11). Consistent with the results obtained with artificial diets, mortality of the adult whiteflies was significantly increased, relative to the control plants transformed with the empty vector, only on plants transformed with RNAi against the two osmoregulation genes and dsRNase genes. However, this effect was underlain by a difference in expression response of the whiteflies on plants and diets to RNAi. Whereas expression of the cognate genes was reduced, often significantly, in whiteflies administered RNAi via artificial diets, RNAi administered in planta had no significant effect on the expression of any of the test genes.

Other aspects of the invention are within the scope of the following numbered paragraphs.
1. A method of using dsRNA to suppress the activity of RNAi-suppressing nuclease genes in the gut of insects.
2. The method of paragraph 1 where the insect is a sap-sucking insect.
3. The method of paragraph 2 where the sap-sucking insect is an aphid, whitefly, mealybug, psyllid, planthopper and leafhopper.
4. The method of paragraph 1 where the dsRNA that suppresses the activity is to an osmoregulation gene.
5. The method of paragraph 4 where the osmoregulation gene is aquaporin AQP1 and/or sucrase SUC1.
6. The method of paragraph 1 where the dsRNA that suppresses the activity is to a nuclease gene.
7. The method of paragraph 6 where the nuclease gene is dsRNase1 and dsRNase2.
8. The method of paragraph 1 where the dsRNA for osmoregulation and nuclease are both used.
9. The method of paragraph 1 where the dsRNA is administered in planta.
10. The method of paragraph 1 where the dsRNA is administered via an artificial diet.

### OTHER EMBODIMENTS

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference. Other embodiments are within the claims.

What is claimed is:

1. A method comprising administering to a phloem sap-feeding insect one or more dsRNAs capable of suppressing activity of one or more RNAi-suppressing nuclease genes expressed in the gut of said insect and one or more dsRNAs capable of suppressing one or more osmoregulatory genes expressed by said insect.

2. The method of claim 1, wherein said phloem sap-feeding insect is an aphid, a whitefly, a psyllid, a mealybug, a planthopper, or a leafhopper.

3. The method of claim 2, wherein is said whitefly is Bemisiatabaci.

4. The method of claim 1, wherein said nuclease is a dsRNase.

5. The method of claim 1, wherein said osmoregulatory gene is an aquaporin or a glucohydrolase of family GH-13.

6. The method of claim 5, wherein said osmoregulatory gene is an aquaporin.

7. The method of claim 5, wherein said glucohydrolase of family GH-13 is a sucrase.

8. The method of claim 1, wherein two dsRNAs that suppress the activity of two dsRNAses, a dsRNA that suppresses an aquaporin, and a dsRNA that suppresses a sucrase are administered to said insect.

9. The method of claim 1, wherein said dsRNAs are administered in planta.

10. The method of claim 1, wherein said dsRNAs are administered in an artificial diet.

11. A plant that is resistant to a phloem sap-feeding insect, wherein said plant comprises one or more dsRNAs capable of suppressing activity of one or more RNAi-suppressing nuclease genes expressed in the gut of said insect and one or more dsRNAs capable of suppressing one or more osmoregulatory genes expressed by said insect.

12. The plant of claim 11, wherein said phloem sap-feeding insect is an aphid, a whitefly, a psyllid, a mealybug, a planthopper, or a leafhopper.

13. The plant of claim 12, wherein is said whitefly is Bemisiatabaci.

14. The plant of claim 11, wherein said nuclease is a dsRNase.

15. The plant of claim 11, wherein said osmoregulatory gene is an aquaporin or a glucohydrolase of family GH-13.

16. The plant of claim 15, wherein said osmoregulatory gene is an aquaporin.

17. The plant of claim 15, wherein said glucohydrolase of family GH-13 is a sucrase.

18. The plant of claim 11, wherein said plant comprises two dsRNAs that suppress the activity of two dsRNAses, a dsRNA that suppresses an aquaporin, and a dsRNA that suppresses a sucrase.

19. A composition comprising one or more dsRNAs capable of suppressing activity of one or more RNAi-suppressing nuclease genes expressed in the gut of said insect and one or more dsRNAs capable of suppressing one or more osmoregulatory genes expressed by said insect.
